(19) United States
(12) Patent Application Publication
(10) Pub. No.: US 2008/0047824 A1
(43) Pub. Date: Feb. 28, 2008

(54) Process for the preparation of polyols and/or polyamines from polyurethanes, polyurethane ureas and polyureas

(76) Inventors: Hartmut Nefzger, Pulheim (DE); Frithjof Hannig, Dusseldorf (DE); Peter Nordmann, Dormagen (DE); Wolfgang Friederichs, Koln (DE)

Correspondence Address: BAYER MATERIAL SCIENCE LLC, 100 BAYER ROAD, PITTSBURGH, PA, 15205, US

(21) Appl. No.: 11/893,453

(22) Filed: Aug. 16, 2007

(30) Foreign Application Priority Data
Aug. 19, 2006 (DE) 102006039057.1

(51) Int. Cl. C07C 209/68; C07C 31/00

(52) U.S. Cl. 204/157.81; 204/157.9

(57) ABSTRACT

The present invention relates to an improved process for the preparation of NCO-reactive components from fully reacted products of the isocyanate polyaddition process, in particular those based on MDI and/or TDI, using reactive solvents in a microwave field.

### FIELD OF THE INVENTION

The present invention relates to an improved process for the preparation of NCO-reactive components, in particular polyols and/or polyamines, from fully reacted products of the isocyanate polyaddition process, in particular those based on MDI and/or TDI, using reactive solvents in a microwave field, and to the use thereof.

### BACKGROUND OF THE INVENTION

It is known that polyethylene terephthalates (PET) can advantageously be glycolyzed by means of microwave radiation (A. Kr\check{z}an. J. Appl. Polymer Science, Vol. 69, p. 1115 (1998)). Unlike the polyurethane ureas under discussion within the scope of the present invention, however, PET is a largely linear, thermoplastic polymer in which a molecular weight degradation is, of course, markedly simpler to achieve than in highly branched systems.

It is further known that polyurethanes prepared by the polyisocyanate polyaddition process can be decomposed into low molecular weight constituents by means of suitable reactive solvents. Such methods are, for example, alcoholysis or glycolysis, according to which polyhydric alcohols such as diols, triols and tetrols are used, and also hydrolysis and aminolysis, which can likewise be used for the liquefaction (degradation) of fully reacted products of the polyisocyanate polyaddition process. It is also possible to use various combinations of these basic reaction types. For example, DE-A 42 17 524 teaches that degradation is carried out first using a glycol-aminoethanol mixture, followed by hydrolytic decomposition.

In DE 195 19 333, polyurethane polyurea waste based on diisocyanatodiphenyl-methane and/or diisocyanatotoluene is reacted in a first step with alcohols at temperatures of from 100 to 260° C. and then treated with o-carboxylic acid triesters for the purpose of reducing the proportion of free aromatic polyamine.

In general, the reaction mixtures in glycolyses, aminolyses, hydrolyses and combinations thereof are heated to elevated temperatures, in some cases to over 250° C., and left at that temperature for a particular dwell time, it being entirely possible, depending on the concrete formulation, for that time to amount even to several days. When comparatively low-boiling reactive solvents, that is to say, for example, water, are used or used concomitantly, it is necessary to work under pressure in order to achieve such high temperatures.

It is desirable in such processes, regardless of the exact formulation, procedure and technical apparatuses used, to improve the space-time yield in order to achieve higher throughputs or, equally, smaller apparatuses with the same throughput, and hence lower investment costs.

In addition, all products prepared in that manner (polyols, polyamines) suffer from far too high a temperature/time load, which can lead to further degradation reactions and accordingly generates low-quality products. An example which may be mentioned is the dehydration of hydroxyl-group-terminated compounds, that is to say the formation of terminal olefin, which means a reduced functionality of these molecules towards NCO groups. This can lead to low-quality materials particularly when used as raw material for polyurethane preparation.

It is therefore also desirable in such processes, regardless of the exact formulation, procedure and technical apparatuses used, to reduce the temperature/time load in order to obtain degradation products (polyols, polyamines) having improved functionality.

Acids or bases or catalysts having a different reaction-accelerating action can be added in order to accelerate the degradation reaction.

However, the use of catalysts having a degradation-accelerating action has the disadvantage that they remain in the reaction product (polyol, polyamine) and must be neutralized so that they are not able to interfere with the reaction when the reaction products are subsequently used as raw material in the preparation of new polyurethane (PUR) products. This means an increased outlay and, in the worst case, products having poor properties.

### SUMMARY OF THE INVENTION

The present invention, therefore, provides a process for the degradation of polyurethanes, polyurethane ureas and polyureas that does not have the above-mentioned disadvantages.

### DETAILED DESCRIPTION OF THE INVENTION

The present invention will now be described for purposes of illustration and not limitation. Except in the operating examples, or where otherwise indicated, all numbers expressing quantities, percentages, OH numbers, functionalities and so forth in the specification are to be understood as being modified in all instances by the term “about.” Equivalent weights and molecular weights given herein in Daltons (Da) are number average equivalent weights and number average molecular weights respectively, unless indicated otherwise.

Surprisingly, it has been found that the degradation (glycolytic, alcoholytic, aminolytic and/or hydrolytic degradation) of the polyurethanes, polyurethane ureas and polyureas can be carried out in the presence of microwave radiation.

The present invention accordingly provides a process for the degradation (liquefaction) of polyurethanes, polyurethane ureas and/or polyureas and hence for the preparation of polyols and/or polyamines, characterized in that the polyurethanes, polyurethane ureas and/or polyureas are mixed with one or more reactive solvents chosen from water, amino compounds having a molecular weight of from 17 to 399 and mono-, di- and poly-functional alcohols having a molecular weight of from 32 to 2500, and this mixture is exposed to microwave radiation, whereby polyols and/or polyamines are formed.

The advantage of the process according to the invention lies both in the fact that reaction-accelerating compounds, such as, for example, acids, bases or catalysts, are not used and in the use thereof, depending on the particular case. For example, it can be advantageous to add catalysts when the recyclate polyol is to be fed to thermal recycling, and the space-time yield of the process is thereby increased.

Microwave radiation in this connection is understood as being the frequency range from 300 MHz to 300 GHz or the wavelength range from 1 m to 1 mm (Römpp, Chemie Lexikon, Thieme Verlag, 9th expanded and revised edition, 1995, p. 2785).

Polyurethanes in this connection are understood as being reaction products of polyisocyanates with at least one further component. Polyurethanes therefore include not only those compounds that contain predominantly or solely urethane groups, but also polyurethane ureas and polyureas. Furthermore, polyurethanes can be both foamed and unfoamed materials and open- or closed-cell polyurethane foams, which can additionally contain also auxiliary substances and additives, for example inorganic or organic fillers, such as, for example, glass fibers, carbon fibers and added ingredients.

Preference is given to polyurethanes that contain as one of the structural components aromatic polyisocyanates, particularly preferably from the family of the diphenylmethane polyisocyanates, toluene polyisocyanates or naphthalene polyisocyanates.

In the process according to the invention, the glycolytic, aminolytic and/or hydrolytic degradation reactions are carried out using microwave radiation, reactive solvents being employed. Reactive solvents are substances that, owing to their chemical structure, are able to react with the functional groups of the polyurethanes. The most important cleavable functional groups of the polyurethanes are ester, carbonate, amide, urethane and urea groups. Consequently, the most important reactive solvents are those which contain hydroxyl and/or amino groups, and also water.

The polyurethanes are preferably used in comminuted form in the process according to the invention, because the rate of dissolution, or the velocity of the degradation reaction, is thereby increased further.

The polyurethanes used are converted into so-called recyclate polyols/polyamines. These can then be used again as raw material in a polyurethane preparation. If a polyurethane is not to be converted into recyclate polyols but merely recycled thermally, partial decomposition (partial degradation) is sufficient. This means that the microwave radiation is discontinued, for example, before full degradation is complete. Instead of recyclate polyols there is then obtained a pumpable mixture of undegraded polyurethane, recyclate polyols and optionally reactive solvents, which mixture can easily be fed to thermal recycling. Because complete decomposition is not necessary, the reaction conditions are adapted accordingly, it being possible to make a saving in terms of reactive solvent and/or to shorten the reaction time.

Energy densities of more than 200 watts/liter are preferred. It is also preferred to irradiate the reaction mixture with microwave energy while at the same time cooling it, so that only a comparatively low reaction temperature is reached despite the high energy input. Compressed air is preferably used for cooling. It is, however, also possible to use other cooling systems, in particular those with a liquid coolant.

The use of microwave devices is not limited to monomode devices, but multimode devices can also be used analogously. Multimode devices are comparable with the generally known domestic devices and have inhomogeneous microwave fields, that is to say so-called hot and cold spots occur inside the microwave chamber owing to this irregular microwave distribution, and this can largely be compensated for by, for example, rotation of a microwave plate.

Monomode devices, on the other hand, have a largely homogeneous microwave field and, owing to a special chamber design, do not exhibit such hot and cold spots.

The microwave radiation can be either monomodal microwave radiation with homogeneous microwave radiation fields or multimodal microwave radiation with heterogeneous microwave radiation fields.

The energy input of the microwave radiation is preferably at least 10 W/l, particularly preferably 50 W/I and very particularly preferably 200 W/l. The polyurethanes, polyurethane ureas and polyureas used are preferably those based on aromatic polyisocyanates. Preferred aromatic polyisocyanates are compounds from the family of the diphenylmethane polyisocyanates, toluene polyisocyanates and naphthalene polyisocyanates.

Polyurethanes, polyurethane ureas and/or polyureas based on aromatic and/or aliphatic polycarboxylic acids are particularly preferably degraded. Suitable polycarboxylic acids are preferably glutaric acid, succinic acid, adipic acid, decanedicarboxylic acid, dodecanedicarboxylic acid, phthalic acid, terephthalic acid, etc.

The substances provided for degradation preferably contain carbonate and/or ether groups.

Temperatures above 90° C. are preferred in the process according to the invention.

The process according to the invention can be carried out not only batchwise but, by using a suitable pump and suitable tubular reactors, also continuously. It is also possible for a plurality of microwave devices to be connected in series or in parallel.

The process can, of course, also be carried out under elevated or reduced pressure. The latter may be advantageous when reactive solvents having a comparatively low boiling point, for example water, ethanolamine or ethylene glycol, are used.

The process is particularly preferably carried out without the use of an inert solvent. In special cases, a solvent can optionally be used concomitantly.

The recyclate polyols/polyamines prepared according to the invention can be used for the preparation of foamed and unfoamed polyurethane, polyurea and polyurethane urea materials. They can also be used as a pumpable fuel.

The invention is to be explained in greater detail by means of the following Examples.

### EXAMPLES

The commercially available monomode microwave device “Discover” from CEM (frequency 2.45 GHz) was used in the Examples. A 100 ml reaction vessel was used in the tests described in greater detail below. The device from CEM is distinguished inter alia by the fact that it is able to produce a comparatively high energy density for microwave devices, which in addition can be maintained at a constant level over a prolonged period as a result of the possibility of simultaneous cooling. The temperature load for the reaction mixture could thus be kept very low.

### Example 1

### According to the Invention

20 parts by weight of comminuted polyurethane urea waste (a waste mixture of BAYDUR 1498 and BAYFLEX 1427, Bayer MaterialScience AG) were mixed with 80 parts by weight of ethylene glycol and heated to reflux temperature in a “Discover” monomode microwave device from CEM (frequency 2.45 GHz) with a power of 300 W, the reaction vessel being cooled with air throughout the test. The reaction mixture was assessed visually in respect of its consistency at 10-minute intervals. After 90 minutes, the reaction mixture was free of solid material and clear. The OH number was determined at 1491 mg KOH/g and the amine number at 34.3 mg KOH/g.
BAYDUR 1498: a polyurethane made of a polyisocyanate of the diphenylmethane diisocyanate group having a NCO value of about 29 wt. % and a polyol component having a hydroxyl number of about 450 mg KOH/g, containing a polyether polyol mixture of polyether polyols containing oligoethylene and oligopropylene oxide units, and aromatic diamine.
BAYFLEX 1427: a polyurethane made of a polyisocyanate of the diphenylmethane diisocyanate group having a NCO value of about 28 wt. % and a polyol component having a hydroxyl number of about 415 mg KOH/g, containing a polyether polyol having oligoethylene and oligopropylene oxide units and low molecular weight diol.

### Example 2

### Comparison Example

20 parts by weight of comminuted polyurethane urea waste (a waste mixture of BAYDUR 1498 and BAYFLEX 1427, Bayer MaterialScience AG) were mixed with 80 parts by weight of ethylene glycol and heated to reflux temperature, with stirring, by means of a heating mantle. The reaction mixture was assessed visually in respect of its consistency at 10-minute intervals. After 90 minutes, the reaction mixture still contained large amounts of solid material. A clear reaction mixture was obtained after 120 minutes. The OH number was 1449 mg KOH/g and the amine number 14.0 mg KOH/g.

### Example 3

### According to the Invention

20 parts by weight of comminuted polyurethane urea waste (a waste mixture of BAYDUR 1498 and BAYFLEX 1427, Bayer MaterialScience AG) were mixed with 80 parts by weight of ethylene glycol and 1.48 parts by weight of 1,1,1-diazabicyclooctane (DABCO) and heated to reflux temperature in a “Discover” monomode microwave device from CEM (frequency 2.45 GHz) with a power of 300 W, the reaction vessel being cooled with air throughout the test. The reaction mixture was assessed visually in respect of its consistency at 10-minute intervals. After 60 minutes, the reaction mixture was free of solid material and clear. The OH number was determined at 1463 mg KOH/g and the amine number at 31.8 mg KOH/g.

### Example 4

### Comparison Example

20 parts by weight of comminuted polyurethane urea waste (a waste mixture of BAYDUR 1498 and BAYFLEX 1427, Bayer MaterialScience AG) were mixed with 80 parts by weight of ethylene glycol and 1.48 parts by weight of 1,1,1-diazabicyclooctane and heated to reflux temperature, with stirring, by means of a heating mantle. The reaction mixture was assessed visually in respect of its consistency at 10-minute intervals. After 60 minutes, the reaction mixture still contained large amounts of solid material. A clear reaction mixture was obtained after 90 minutes. The OH number was determined at 1463 mg KOH/g and the amine number at 31.8 mg KOH/g.

### Examples 5, 6, 7:

These examples were carried out according to Example 3 but with different amounts of catalyst (see Table 1).

### Example 8

### Comparison Example

This example was carried out according to Example 4 with an amount of catalyst of 0.36 part by weight.

The results of the examples are summarized in Table 1.

TABLE 1

| | | Reaction |
|---|---|---|
| | DABCO | time |
| Example | [parts by wt.] | [min] |
| 1 | 0 | 90 |
| C2 | 0 | 120 |
| 3 | 1.48 | 60 |
| C4 | 1.48 | 90 |
| 5 | 1.96 | 60 |
| 6 | 0.99 | 60 |
| 7 | 0.36 | 60 |
| C8 | 0.36 | 75 |

The ratios used were in each case 20 parts by weight of polyurethane urea and 80 parts by weight of ethylene glycol. The indicated amount of 1,1,1-diazabicyclooctane (DABCO) was used as catalyst in each case. The reaction was carried out under reflux conditions in all cases. The necessary reaction time to obtain a clear reaction mixture is indicated in the column “Reaction time” in Table 1. The examples according to the invention were carried out according to the procedure described in Examples 1 and 3 using a microwave device.

The results of the tests listed in Table 1 show that, with the same formulation, the reactions proceeded faster when the reaction mixtures were exposed to microwave radiation.

The savings in terms of time that were achieved under the chosen conditions varied within an order of magnitude of about 30%.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the degradation of one of a polyurethane, a polyurethane urea and/or polyurea, comprising:
mixing the one of a polyurethane, a polyurethane urea and/or polyurea with one or more reactive solvents selected from the group consisting of water, amino compounds having a molecular weight of from about 17 to about 399 and mono-, di- and poly-functional alcohols having a molecular weight of from about 32 to about 2500 to form a mixture; and
exposing the mixture to microwave radiation to form a polyol and/or a polyamine.

2. The process according to claim 1, wherein the microwave radiation is monomodal microwave radiation with homogeneous microwave radiation fields.

3. The process according to claim 1, wherein the microwave radiation is multimodal microwave radiation with heterogeneous microwave radiation fields.

4. The process according to claim 1, wherein the energy input of the microwave radiation is at least about 10 W/l.

5. The process according to claim 1, wherein the energy input of the microwave radiation is at least about 50 W/l.

6. The process according to claim 1, wherein the energy input of the microwave radiation is preferably more than about 200 W/l.

7. The process according to claim 1, wherein the polyurethane, polyurethane urea and/or polyurea is based on an aromatic polyisocyanate.

8. The process according to claim 7, wherein the aromatic polyisocyanate is selected from the group consisting of diphenylmethane polyisocyanates, toluylene polyisocyanates and naphthylene polyisocyanates.

9. The process according to claim 1, wherein the polyurethane, polyurethane urea and/or polyurea comprises one or more aromatic and/or aliphatic polycarboxylic acids.

10. The process according to claim 9, wherein the one or more aromatic and/or aliphatic polycarboxylic acids are selected from the group consisting of glutaric acid, succinic acid, adipic acid, decanedicarboxylic acid, dodecanedicarboxylic acid, phthalic acid and terephthalic acid.

11. The process according to claim 1, wherein the polyurethane, polyurethane urea and/or polyurea contains carbonate groups and/or ether groups.

12. The process according to claim 1, wherein the process is carried out under elevated pressure.

13. The process according to claim 1, wherein the process is carried out at temperatures above about 90° C.

14. In a process for the preparation of one of foamed and unfoamed polyurethane, polyurethane urea or polyurea materials, the improvement comprising including a polyol and/or polyamine prepared by the process according to claim 1.
